(19) United States
(12) Patent Application Publication
(10) Pub. No.: US 2023/0226099 A1
(43) Pub. Date: Jul. 20, 2023

(54) INHALABLE FORMULATION

(71) Applicant: AALTO UNIVERSITY FOUNDATION SR, Aalto (FI)

(72) Inventors: Christopher JONKERGOUW, Aalto (FI); Nurcin UGUR, Aalto (FI); Markus LINDER, Aalto (FI); Janne RAULA, Aalto (FI); Esko KAUPPINEN, Aalto (FI); Ekaterina OSMEKHINA, Aalto (FI)

(21) Appl. No.: 17/917,346

(22) PCT Filed: Apr. 7, 2021
(86) PCT No.: PCT/FI2021/050254

§ 371 Date: Oct. 6, 2022

(30) Foreign Application Priority Data
Apr. 7, 2020 (FI) 20205368

(51) Int. Cl. A61K 31/724; A61K 31/407; A61K 31/7036; A61K 31/5383; A61K 9/00; A61K 9/16; A61P 31/04

(52) U.S. Cl. CPC A61K 31/724; A61K 31/407; A61K 31/7036; A61K 31/5383; A61K 9/0075; A61K 9/0078; A61K 9/1623; A61P 31/04

(57) ABSTRACT

This invention relates to inhalable formulation comprising a macrocyclic, cavity-containing compound as a biologically active ingredient. The present invention relates also to an inhalable formulation comprising a macrocyclic, cavity-containing compound as a biologically active ingredient for use in a method of treating an infection in a subject. In addition, the present invention relates to a method of treating an infection in a subject by administering an inhalable formulation comprising a macrocyclic, cavity-containing compound as a biologically active ingredient to the subject. The inhalable formulation comprising a macrocyclic, cavity-containing compound as a biologically active ingredient is suitable for the treatment of pulmonary or systemic infections caused by Gram-positive or Gram-negative bacteria.

### FIELD OF THE INVENTION

This invention relates to inhalable formulation comprising a macrocyclic, cavity-containing compound as a biologically active ingredient. The present invention relates also to an inhalable formulation comprising a macrocyclic, cavity-containing compound as a biologically active ingredient for use in treating an infection in a subject. In addition, the present invention relates to a method of treating an infection in a subject by administering an inhalable formulation comprising a macrocyclic, cavity-containing compound as a biologically active ingredient to the subject. The inhalable formulation comprising a macrocyclic, cavity-containing compound as a biologically active ingredient is suitable for the treatment of infections caused by Gram-positive bacteria, such as Staphylococcus aureus or Gram-negative bacteria, such as Pseudomonas aeruginosa, Acinetobacter baumannii, Klebsiela pneumoniae and other Enterobacteriaceae.

### BACKGROUND OF THE INVENTION

According to the World Health Organization's report in 2018 lower respiratory infections remained one of the deadliest contagious disease, being the fourth top cause of death worldwide in 2016. The report indicates the urgency to develop more efficient treatment methods for respiratory infections. Bacterial infection of the lower respiratory tract is associated with high morbidity and mortality rates especially for hospitalized patients and for patients with cystic fibrosis (CF). Ventilator-associated pneumonia (VAP) caused by Pseudomonas aeruginosa is one of the most common lung infections in hospitalized patients. Treatment of such lung infections is challenging since systemic antibiotic administration results in lower concentrations in lung tissue.

Pulmonary drug delivery has attracted remarkable interest in recent years for the treatment of local or systemic infections. In fact, the lung mucosa has proved particularly attractive for systemic administration, given the large alveolar area exposed for drug absorption (approximately 100 m²), and thin alveolar-vascular epithelium (0.1-0.2 μm) that permits rapid absorption and avoids the first-pass effect compared to injections.

Pharmaceutical powders can be delivered to the lungs by using three different types of devices i.e., by nebulizers, pressurized metered dose inhalers (pMDI) and dry powder inhalers (DPI). These devices emit an aerosol of particles or droplets and differ in the technology used for aerosol delivery. Nebulizers are not portable, therefore both pMDIs and DPIs are more commonly used. The main difference between pMDIs and DPIs is that in the former the drug is dispersed in a liquid propellant under pressure, while the DPIs contain dry powders. Liquid based drug solutions require further components in order to stabilize the drug. Meanwhile, dry powders require much less stabilizers compared to liquid drug systems for MDIs. Therefore, the DPI systems do not encounter the stability problems usually presented by suspensions. The technology of the inhaler device plays a very important role in the efficacy of the treatment, since it can influence aerosol distribution within the lung.

The present invention provides highly dispersible and stable inhalable formulations comprising a macrocyclic, cavity-containing compound as a biologically active ingredient to target both local and systemic infections in order to reduce toxicity caused by bacteria and/or to increase the treatment efficacy.

The publications and other materials referred herein to illuminate the background of the invention, and in particular, cases to provide additional details respecting the practice, are incorporated by reference.

### BRIEF DESCRIPTION OF THE INVENTION

The present invention relates to inhalable formulations comprising a macrocyclic cavity-containing compound as a biologically active ingredient. The present invention relates also to inhalable formulations comprising a macrocyclic cavity-containing compound and an antimicrobial agent as biologically active ingredients. The present invention relates also to an inhalable formulation comprising a macrocyclic, cavity-containing compound as a biologically active ingredient for use in inhibiting and/or treating and/or preventing a microbial infection in a subject. In addition, the present invention relates to an inhalable formulation comprising a macrocyclic, cavity-containing compound and an antimicrobial agent as biologically active ingredients for use in inhibiting and/or treating and/or preventing a microbial infection in a subject. Further, the present invention relates to a method of inhibiting and/or treating and/or preventing a microbial infection in a subject by administering an inhalable formulation comprising a macrocyclic, cavity-containing compound as a biologically active ingredient to the subject. The present invention relates also to a method of inhibiting and/or treating and/or preventing a microbial infection in a subject by administering an inhalable formulation comprising a macrocyclic, cavity-containing compound and an antimicrobial agent as biologically active ingredients to the subject.

The inhalable formulation may be delivered utilizing any applicable device known in the art, such as a nebulizer or a dry powder inhaler (DPI), for example.

The formulations according to the invention provide a safe, non-toxic pulmonary delivery of a macrocyclic cavity-containing compound as a biologically active ingredient. In addition, the formulations of the present invention provide physiologically stable and compatible pulmonary delivery of a macrocyclic cavity-containing compound as a biologically active ingredient. The formulations according to the invention provide also a safe, non-toxic pulmonary delivery of a combination of a macrocyclic cavity-containing compound and an antimicrobial agent as biologically active ingredients. In addition, the formulations of the present invention provide physiologically stable and compatible pulmonary delivery of a combination of a macrocyclic cavity-containing compound and an antimicrobial agent as biologically active ingredients. The formulations according to the invention are suitable for the treatment of acute local microbial infections. The formulations according to the invention are suitable for the treatment of acute systemic microbial infections. The formulations according to the invention are suitable for the treatment of sub-acute local microbial infections. The formulations according to the invention are suitable for the treatment of sub-acute systemic microbial infections. The formulations according to the invention are suitable for the treatment of the chronic local microbial infections. The formulations according to the invention are suitable for the treatment of systemic microbial infections.

The objects of the invention are achieved by compounds, uses and methods characterized by what is stated in the independent claims. The preferred embodiments of the invention are disclosed in the dependent claims.

### BRIEF DESCRIPTION OF THE DRAWINGS

FIG. 1 shows SEM images of a formulation comprising pillar[5]arene (13%), trehalose, leucine and sodium citrate.

FIG. 2 shows SEM images of a formulation comprising α-cyclodextrin (13%), trehalose, leucine and sodium citrate.

FIG. 3 shows SEM images of a formulation comprising cucurbit[6]uril (15%), trehalose, leucine and sodium citrate.

FIG. 4 shows the effects of a formulation comprising pillar[5]arene (36%), trehalose, leucine and sodium citrate on the pyocyanin toxin production in P. aeruginosa after 24 hours.

FIG. 5 shows the effects of a formulation comprising pillar[5]arene (36%), trehalose, leucine and sodium citrate on human A549 lung cells, infected with P. aeruginosa, for 24 hours.

FIG. 6 shows SEM images of a formulation comprising 18-crown-6 (12.5%), colistin (12.5%), trehalose, leucine and sodium citrate.

FIG. 7 shows SEM images of a formulation comprising 15-crown-5 (12.5%), amikacin (12.5%), raffinose, leucine and sodium citrate.

FIG. 8 shows SEM images of a formulation comprising γ-cyclodextrin (12.5%), ciprofloxacin (12.5%), mannitol, leucine and sodium citrate.

FIG. 9 shows the lung neutrophil infiltration and inflammation score of BALB/c mice lung tissue, administered with different dosages of P[5]a for 24 hours. Two different administration methods were use, intravenously (i.v.) or intranasally (i.n.). Pathological changes were graded according to severity (P=Present; 1=Minimal; 2=Mild; 3=Moderate). The score indicates a good tolerability of P[5]a, both by i.n. and i.v. administration.

FIG. 10 shows a schematic representation of the structural features that lead to a dual mechanism of action of P[5]a. a) Highlighted in orange is the hydrophobic core of the structure, with a cavity size of 4.6 Å, which binds the signalling molecule. Highlighted in blue are the positively charged amino groups that interact with the negatively charged surface of the cell membrane. b) Graphic representation of the effects of the proposed dual mechanisms of P[5]a on P. aeruginosa.

FIG. 11 shows the interaction of P[5]a with lipopolysaccharides of P. aeruginosa, strain PA10. a, Analytical ultracentrifuge sedimentation velocity analysis of P[5]a alone shows steady sedimentation profile at 305 nm. b, Analytical ultracentrifuge sedimentation velocity analysis of LPS alone shows no detectable sedimentation profile at 305 nm, which shows that at 305 nm, the sedimentation of P[5]a.is only followed. c, Analytical ultracentrifuge sedimentation velocity analysis of P[5]a together with LPS shows a very rapid and varying sedimentation profile at 305 nm, showing P[5]a-LPS complexes (indicated by arrows). This is followed by a large amount of unbound P[5]a, sedimenting slower (indicated by arrow). d, Molecular weight distribution of sedimentation profiles, strong interaction between 135 μM P[5]a and 35 μM LPS in UV absorbance at 305 nm. A clear initial peak of unbound P[5]a can be observer at low molecular weight (2260 Da), followed by a long and polydisperse collection of peaks, ranging from low molecular weight (S), to very high molecular weight (60.000 kDa) See Example 2.

FIG. 12 shows the effect of P[5]a on the formation of biofilm in the pathogenic Gram-negative bacteria Pseudomonas aeruginosa. ***P<0.001; **P<0.01; *P<0.05. (Example 3).

FIG. 13 shows the effect of P[5]a on the formation of biofilm in “Superbug” strains of the pathogenic Gram-negative bacteria Pseudomonas aeruginosa and Acinetobacter baumannii. These strains are highly resistant to common utilized treatments. Resistance profiles and detailed strain information are provided in Table 2. ***P<0.001; **P<0.01; *P<0.05.

FIG. 14 shows the protein subcellular localization of core enriched gene products of P[5]a treated P. aeruginosa (PAO1), shows the largest changes in proteins targeted towards the extracellular space (secretion), outer membrane vesicles and the outer membrane.

FIG. 15 shows how P[5]a enhances the penetration of coadministered antibiotics in Pseudomonas aeruginosa PAO1. The coadministration of P[5]a with the antibiotics Aztreonam (a), Cefepime (b), Meropenem (c) and Tobramycin (d) greatly slowed the development of resistance by the pathogen to the respective antibiotic treatment. MIC values were categorized in three groups according to the Clinical and Laboratory Standards Institute: susceptible, intermediate susceptible and resistant. Cefepime reached 128 μg/ml after four days, which was the highest concentration of antibiotic included.

FIG. 16 shows how P[5]a enhances the penetration and efficacy of coadministered antibiotics Amikacin (a), Cefepime (b), Ceftazidime (c) and Meropenem (d) in MDR resistant clinical isolates.

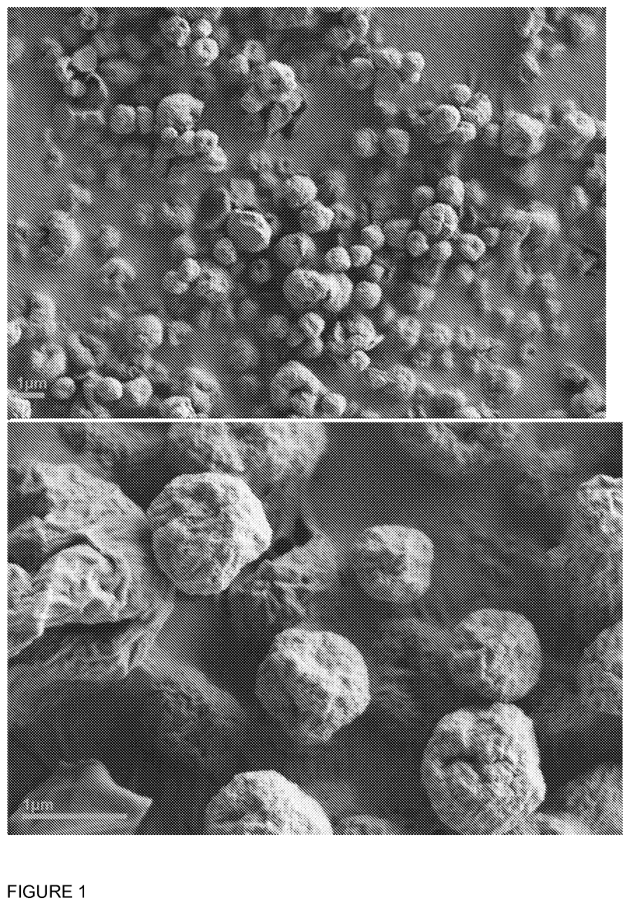

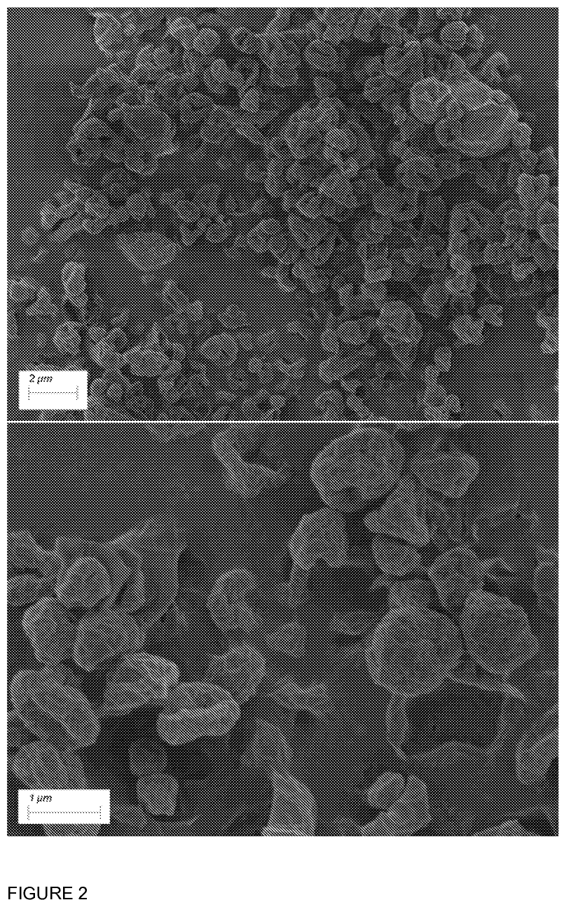

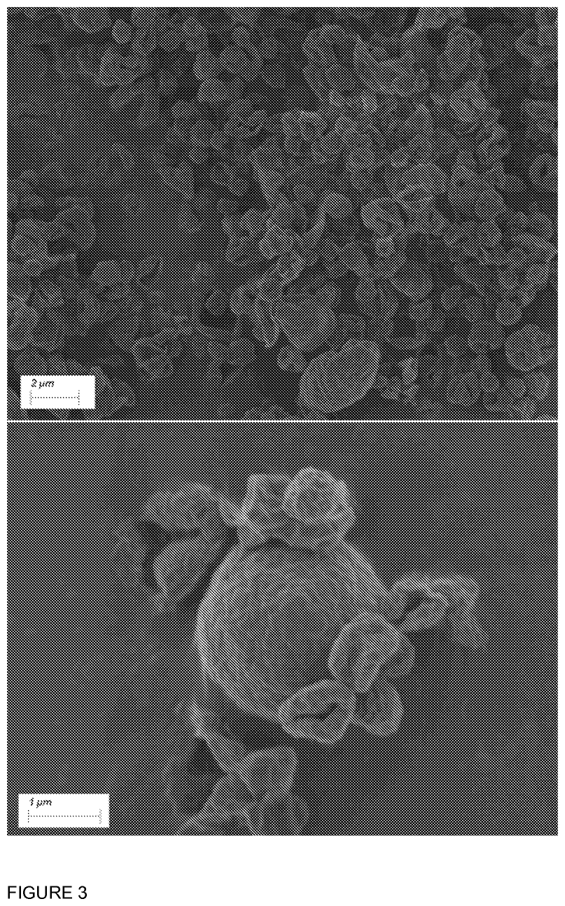

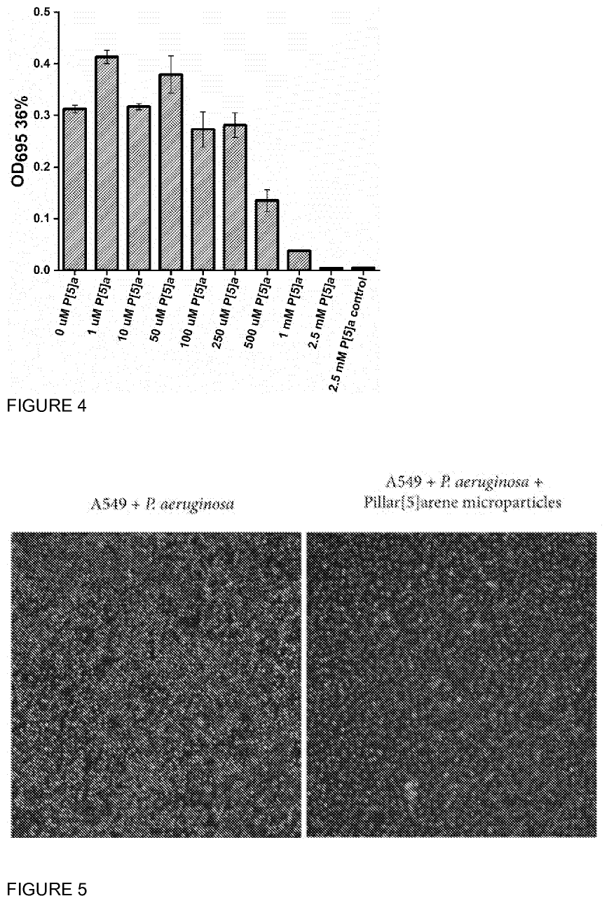

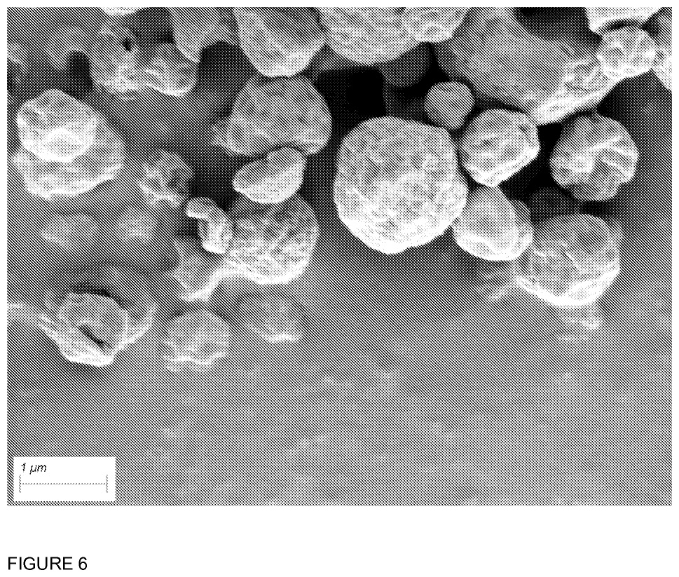

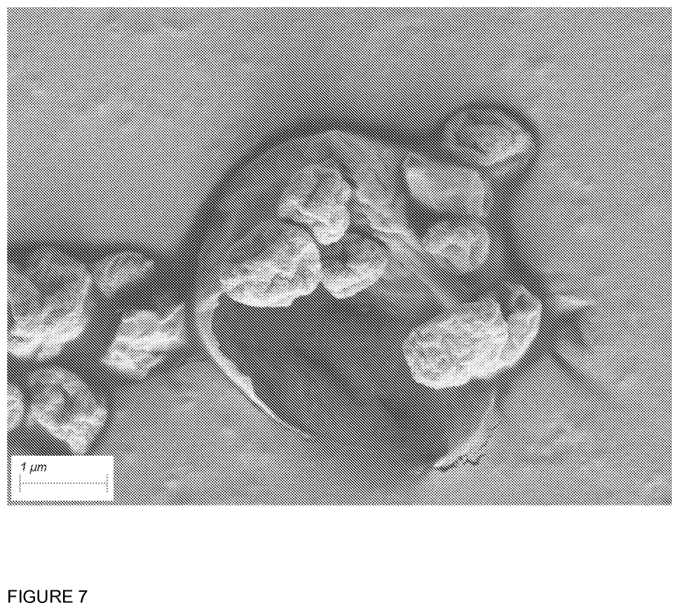

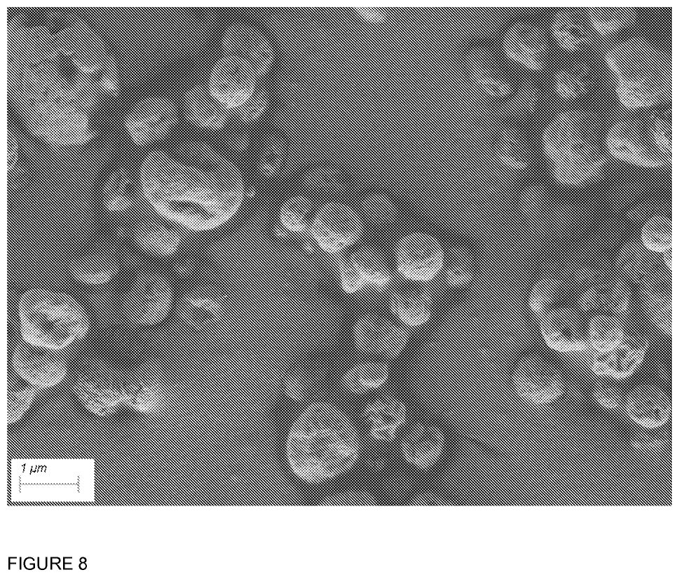

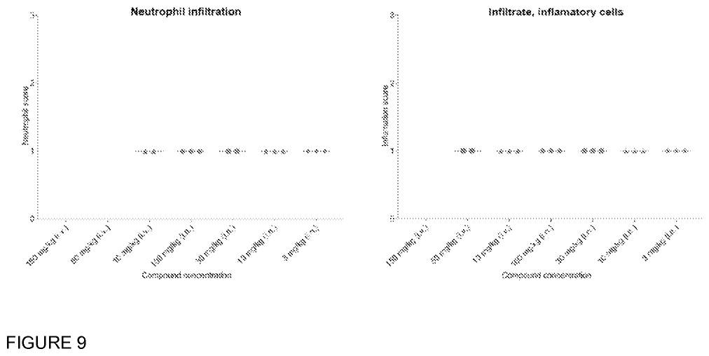

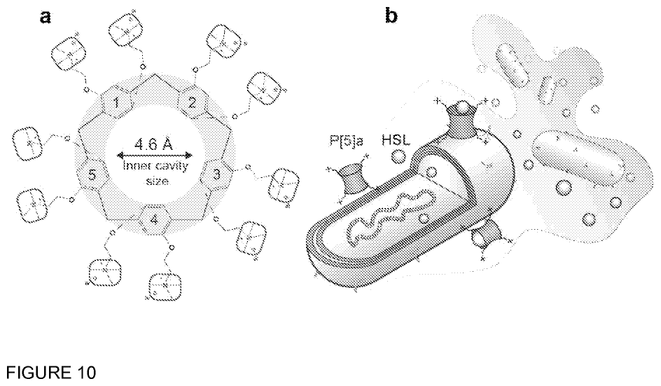

### DETAILED DESCRIPTION OF THE INVENTION

Unless otherwise specified, the terms used in the description and claims have the meanings known to a person skilled in the art.

In the present specification, the term “macrocyclic cavity-containing compound” refers to an organic cyclic compound forming cylindrical structure providing a cavity for host-guest interaction. The macrocyclic cavity-containing compounds have been found to prevent or treat a microbial signaling molecule dependent and/or mediated microbial infection by binding the microbial signaling molecule by non-covalent host-quest bonding. In addition, the macrocyclic cavity-containing compounds bind specific components of the biofilm matrix. As a result, the microbes stop or reduce the production of one or several of toxins, biofilms and other virulence factors. Thus, the macrocyclic cavity-containing compounds act as virulence inhibitors and this mode of action differs significantly from antibiotics, which either inhibit growth of the pathogens or kill the pathogens. The macrocyclic cavity-containing compounds have no negative growth effects on microbes. Thus, the microbial cells are not under a pressure for survival and are less likely to gain and/or build up resistance. The host-guest binding of a macrocyclic cavity-containing compound and a microbial signalling molecule is solely an extracellular process. The macrocyclic cavity-containing compounds are too large to enter the microbial cells, which further reduces the chances of resistance development in microbes. The macrocyclic cavity-containing compounds do not affect the viability of the microbial cells. Further, they do not affect the viability of the animal cells. Thus, their administration into the lungs is not harmful to lung cells.

The microbial signalling molecules or the quorum sensing (QS) molecules, are a group of small diffusible molecules, which bacteria can sense and release and which are utilized as a form of communication. In bacteria, especially in many Gram-negative bacteria, the signalling molecules regulate a wide variety of virulence associated factors, such as biofilm formation, the production of exotoxins and surfactants, motility, and nutrient scavenging molecules, as a means to increase chances of successful infections. In one embodiment, the microbial signalling molecule is a microbial quorum sensing signal molecule. In one embodiment, the microbial signalling molecule or the microbial quorum sensing signal molecule is homoserine lactone (HSL) and/or N-acyl-homoserine lactone (AHL). In one embodiment, the carbon chain of the HSL or the AHL has a length of 4 to 18 or 6 to 14 carbon atoms. In one embodiment, the carbon chain of the HSL or the AHL is linear. In one embodiment, the carbon chain of the HSL or the AHL is branched. In one embodiment, the macrocyclic cationic cavity-containing compound is also able to interact with extracellular DNA in the extrapolymeric substance.

The macrocyclic cavity-containing compounds have been found to enhance the efficacy of conventional antimicrobial agents when they administered together or in combination.

In the inhalable formulations of the present invention, the macrocyclic cavity-containing compound acts as a biologically active ingredient. In one embodiment, the macrocyclic cavity-containing compound acts as a pharmaceutically active ingredient. In one embodiment, the biological activity refers to pharmaceutical activity. In one embodiment, the biological activity refers to virulence suppressing activity.

In addition, the macrocyclic cavity-containing compounds have good stability and are easily dissolved, and even stable, in water. Thus, these compounds can be applied in a wide variety of environments.

Examples of such macrocyclic cavity-containing compounds are pillararenes, cucurbiturils, cyclodextrins, crown ethers, and calixarenes. In the present invention, on one hand, the delivery of a macrocyclic cavity-containing compound to the local infection site in lungs improves the treatment, and higher concentration at the site of infection with relatively low doses of the active compound can be achieved compared to the systemic treatments. The benefit is that the lower doses prevent from potential adverse drug reactions. On the other hand, in the case of a targeted systemic exposure through the pulmonary route, the inhalable formulations can provide higher drug absorption compared to the injections or orally administered dosage forms. Formulations for pulmonary delivery also provide a non-invasive method of administration that can be used both in primary and secondary care.

The inhalable formulation is delivered as an aerosol or as an inhalable dry powder. The inhalable formulations can be delivered with nebulizers or with dry powder inhalers (DPI), for example. Without nebulization or dry powder formulations, a macrocyclic cavity-containing compound is difficult to deliver directly to the lungs.

In one embodiment, the macrocyclic cavity-containing compound is selected from pillararenes, cucurbit urils, crown ethers, cyclodextrins, calixarenes, and/or salts thereof. In one embodiment, the macrocyclic cavity-containing compound is selected from pillararenes and/or salts thereof. In one embodiment, the macrocyclic cavity-containing compound is selected from pillar[5]arenes or salts thereof. In one embodiment, the pillar[5]arene is 4,9,14,19,24,26,28,30,32,34-Deca[2-(trimethylaminio)ethoxy]hexacyclo[21.2.2.2^{3,6}.2^{8,11}.2^{13,16}.2^{18,21}]pentatriaconta1(25),3,5,8,10, 13,15,18,20,23,26,28,30,32,34-pentadecaene 10bromide. In one embodiment, the macrocyclic cavity-containing compound is selected from resorcin arenes and/or salts thereof. In one embodiment, the macrocyclic cavity-containing compound is resorcin[4]arene or a salt thereof. In one embodiment, the macrocyclic cavity-containing compound is selected from crown ethers. In one embodiment, the crown ether is 18-crown-6 (1,4,7,10,13,16-Hexaoxacyclooctadecane). In one embodiment, the crown ether is 15-crown-5 (1,4,7,10,13-Pentaoxacyclopentadecane). In on embodiment, the macrocyclic cavity-containing compound is selected from cucurbit urils. In one embodiment, the cucurbit uril is cucurbit[6]uril. In one embodiment, the macrocyclic cavity-containing compound is selected from cyclodextrins or salts thereof. In one embodiment, the macrocyclic cavity-containing compound is selected from alpha-cyclodextrins, gamma-cyclodextrins or salts thereof. In one embodiment, the macrocyclic cavity-containing compound is alpha-cyclodextrin or a salt thereof. In one embodiment, the macrocyclic cavity-containing compound is gamma-cyclodextrin or a salt thereof. In one embodiment, the macrocyclic cavity-containing compound is selected from calixarenes or salts thereof. In one embodiment, the calixarene is 4-sulfocalix[4]arene. In one embodiment, the macrocyclic cavity-containing compound is selected from a group comprising a pillar[5]arene, a resorcin[4]arene, 18-crown-6, 15-crown-5, cucurbit[6]uril, an alpha-cyclodextrin, a gamma-cyclodextrin and 4-sulfocalix[4]arene.

In one embodiment, the inhalable formulation may comprise also an antimicrobial agent. The antimicrobial agent can be a β-lactam such as a penicillin derivative, a cephalosporin, a carbepenem and a β-lactamase inhibitor, an aminoglycoside, a fluoroquinolone, a macrolide, a tetracycline, novobiosin, chloramphenicol, ethidium bromide or colistin.

In one embodiment, the antimicrobial agent is a β-lactam antibiotic or a combination of β-lactam antibiotics. In one embodiment, the β-lactam antibiotic is a penicillin derivative. In one embodiment, the penicillin derivative is piperacillin or ticarcillin. In one embodiment, the β-lactam antibiotic is aztreonam. In one embodiment, the β-lactam antibiotic is a β-lactamase inhibitor. In one embodiment, the β-lactamase inhibitor is tazobactam or clavulanic acid. In one embodiment, the β-lactam antibiotic is a combination of a penicillin derivative and a β-lactamase inhibitor. In one embodiment, the combination of a penicillin derivative and a β-lactamase inhibitor is a combination of pipercacillin and tazobactam or a combination of ticarcillin and clavulanic acid. In one embodiment, the combination of a β-lactamase inhibitor and a β-lactam antibiotic is a combination of imipenem and relebactam with cilas-tatin.

In one embodiment, the β-lactam antibiotic is a cephalosporin. In one embodiment, the cephalosporin is cefepime, ceftazidime, cefoperazone, cefpirome, ceftriaxone or ceftobiprole. In one embodiment, the β-lactam antibiotic is a carbepenem. In one embodiment, the carbepenem is imipenem, meropenem, ertapenem, doripenem, panipenem, biapenem or tebipenem.

In one embodiment, the antimicrobial agent is an aminoglycoside. In one embodiment, the aminoglycoside is kanamycin, amikacin, tobramycin, dibekacin, gen-tamycin, sismycin, netilmycin, neomycin B, neomycin C, neomycin E, streptomycin, or plazomycin.

In one embodiment, the antimicrobial agent is a fluoroquinolone. In one embodiment, the fluoroquinolone is ciprofloxacin, levofloxacin, garenoxacin, gatifloxacin, gemifloxacin, norfloxacin, ofloxacin or moxifloxacin.

In one embodiment, the antimicrobial agent is a macrolide. In one embodiment, the macrolide is azithromycin.

In one embodiment, the antimicrobial agent is polymyxin. In one embodiment, the polymyxin is polymyxin B or colistin. In one embodiment, the polymyxin is colistin.

In one embodiment, the macrocyclic cavity-containing compound is a pillararene and the antimicrobial agent is colistin. In one embodiment, the macrocyclic cavity-containing compound is a pillararene and the antimicrobial agent is a fluoroquinolone, such as levofloxacin. In one embodiment, the macrocyclic cavity-containing compound is pillararene, such as pillar[5]arene and the fluoroquinoline is ciprofloxacin. In one embodiment, the macrocyclic cavity-containing compound is a pillararene and the antimicrobial agent is a β-lactam antibiotic, such as cephalosporin. In one embodiment, the pillararene is pillar[5]arene and the β-lactam antibiotic is cephalosporin. In one embodiment, the pillararene is pillar[5]arene and the β-lactam antibiotic is cefepime. In one embodiment, the macrocyclic cavity-containing compound is a pillararene, such as pillar[5]arene and the antimicrobial agent is a β-lactam antibiotic, such as aztreonam. In one embodiment, the macrocyclic cavity-containing compound is a pillararene, such as pillar[5]arene, and the anti antimicrobial agent is an aminoglycoside, such as tobramycin. In one embodiment, the macrocyclic cavity-containing compound is a pillararene, such as pillar[5]arene, and the antimicrobial agent is meropenem. In one embodiment, the macrocyclic cavity-containing compound is a pillararene, such as pillar[5]arene, and the antimicrobial agent is a macrolide, such as azithromycin.

In one embodiment, the macrocyclic cavity-containing compound is a crown ether and the antimicrobial agent is a polymyxin. In one embodiment, the crown ether is 18-crown-6 and the polymyxin is colistin. In one embodiment, the macrocyclic cavity-containing compound is a crown ether and the antimicrobial agent is an aminoglycoside. In one embodiment, the crown ether is 15-crown-5 and the aminoglycoside is amikacin.

In one embodiment, the macrocyclic cavity-containing compound is a cyclodextrin and the antimicrobial agent is a fluoroquinolone. In one embodiment, the cyclodextrin is γ-cyclodextrin and the fluoroquinoline is ciprofloxacin. In one embodiment, the macrocyclic cavity-containing compound is a cyclodextrin and the antimicrobial agent is colistin. In one embodiment, the macrocyclic cavity-containing compound is a cyclodextrin and the antimicrobial agent is a fluoroquinolone, such as levofloxacin. In one embodiment, the macrocyclic cavity-containing compound is cyclodextrin and the fluoroquinoline is ciprofloxacin. In one embodiment, the macrocyclic cavity-containing compound is a cyclodextrin and the antimicrobial agent is a β-lactam antibiotic, such as aztreonam. In one embodiment, the macrocyclic cavity-containing compound is a cyclodextrin and the anti antimicrobial agent is an aminoglycoside, such as tobramycin. In one embodiment, the macrocyclic cavity-containing compound is a cyclodextrin and the antimicrobial agent is a macrolide, such as azithromycin.

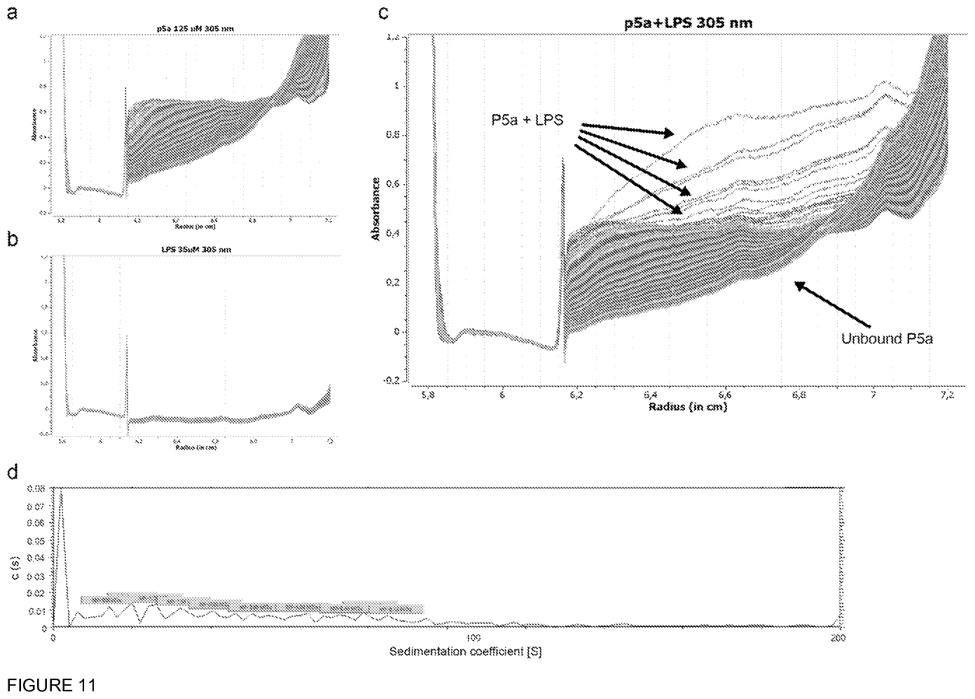

The macrocyclic cavity-containing compounds were found to have a dual mechanism of action on Gram-negative micro-organisms. Firstly, they were found to attenuate the virulence through binding of microbial signaling molecules inside the inner cavity of the molecule. Secondly, they were found to sensitize bacterial outer membrane by the positively charged functional side groups. Specifically, pillar[5]arene, a macrocyclic cavity-containing compound, was found to attenuate virulence through binding of homoserine lactone (HSL) signaling molecules inside its inner cavity and to sensitize the bacterial outer membrane by binding the lipopolysaccharides (LPSs) of the bacterial outermembrane by its positively charged functional side groups. The strong interaction of a macrocyclic cavity-containing compound, P[5]a, with lipopolysaccharides of P. aeruginosa, strain PA10 is shown in FIG. 11 by the analytical ultracentrifuge analysis. The results indicate that P[5]a can interact with multiple LPS units, and might even act as a scaffold for higher-order structures of large molecular weight.

The dual mechanism of action strengthens the ability of the macrocyclic cavity-containing compounds to treat infections caused by Gram-negative bacteria by themselves. In addition, the dual mechanism of action strengthens the ability of the macrocyclic cavity-containing compounds to treat infections caused by Gram-negative bacteria with antibiotics, even those having intracellular targets. It was found that the macrocyclic cavity-containing compound and the antibiotic had a synergistic effect on an infection caused by a gram-negative bacterium. Without wishing to be bound by any theory, the dual mechanism of action of the macrocyclic cavity-containing molecule on Gram-negative bacteria forms the basis for the effective sensitization of a microbe towards an antimicrobial agent and thus leads to the reduction of the amount of an antimicrobial agent needed to prevent or inhibit the growth of the microbe in a subject or to kill the pathogenic microbe in a subject, when a macrocyclic cavity-containing compound and an antimicrobial agent are admistered in combination.

In the present invention, the macrocyclic cavity-containing compounds were found to function with a wide variety of antimicrobial agents/antibiotics. The macrocyclic cavity-containing coumpounds and antimicrobial agents were found to have synergistic effects, such as a drop in minimal inhibitory concentrations and a significantly reduced resistance build-up of pathogens to the antimicrobial agents and/or antibiotics. The effects were found with antimicrobial agents from a diverse range of classes and mechanisms.

The inhalable formulation of the present invention comprises a macrocyclic cavity-containing compound in a biologically effective amount. In one embodiment, the inhalable formulation comprises a macrocyclic cavity-containing compound in a pharmaceutically effective amount. In one embodiment, the inhalable formulation comprises a macrocyclic cavity-containing compound in an amount having virulence suppressing activity.

The inhalable formulation may comprise an administration dose of a macrocyclic cavity-containing compound of about 0.1 mg/kg to about 500 mg/kg. In one embodiment, the dose of a macrocyclic cavity-containing compound is about 1 mg/kg to about 100 mg/kg. In one embodiment, the dose of a macrocyclic cavity-containing compound is about 1 mg/kg to about 50 mg/kg or about 1 mg/kg to about 40 mg/kg. In one embodiment, the dose of a macrocyclic cavity-containing compound is about 1 mg/kg to about 12 mg/kg. The dose can be administered once, twice, three times or four times a day up to a daily dose of 2000 mg/kg. In one embodiment, the daily dose is up to 160 mg/kg or up to 48 mg/kg. In one embodiment, the inhalable formulation may comprise an administration dose of pillar[5]arene of about 0.1 mg/kg to about 500 mg/kg. In one embodiment, the dose of pillar[5]arene is about 1 mg/kg to about 100 mg/kg. In one embodiment, the dose of pillar[5]arene is about 1 mg/kg to about 50 mg/kg or about 1 mg/kg to about 40 mg/kg. In one embodiment, the dose of pillar[5]arene is about 1 mg/kg to about 12 mg/kg. The dose can be administered once, twice, three times or four times a day up to a daily dose of 2000 mg/kg. In one embodiment, the daily dose is up to 160 mg/kg or up to 48 mg/kg.

In one embodiment, the dose of the macrocyclic cavity-containing compound in the formulations of the invention ranges from 0.1% to 99.9%, 1% to 99%, 5% to 95%, 5% to 85%, 5% to 75%, 5% to 65%, 5% to 55%, 5% to 45%, 5% to 35%, 5% to 25%, 5% to 15% based on the weight percentage of the formulation. In one embodiment, the dose of the macrocyclic cavity-containing compound in the formulations of the invention ranges from 10% to 90%, 10% to 80%, 10% to 70%, 10% to 60% or 10% to 50% based on the weight percentage of the formulation. In one embodiment, the dose of the macrocyclic cavity-containing compound in the formulations of the invention ranges from 20% to 90%, 20% to 80%, 20% to 70%, 20% to 60% or 20% to 50% based on the weight percentage of the formulation. In one embodiment, the dose of the macrocyclic cavity-containing compound in the formulations of the invention ranges from 30% to 90%, 30% to 80%, 30% to 70%, 30% to 60% or 30% to 50% based on the weight percentage of the formulation. In one embodiment, the dose of the macrocyclic cavity-containing compound in the formulations of the invention ranges from 40% to 90%, 40% to 80%, 40% to 70%, 40% to 60% or 40% to 50% based on the weight percentage of the formulation.

In one embodiment, the dose of an antimicrobial agent in the formulations of the invention ranges from 5% to 95%, 5% to 85%, 5% to 75%, 5% to 65%, 5% to 55%, 5% to 45%, 5% to 35%, 5% to 25%, 5% to 15% based on the weight percentage of the formulation.

In one embodiment, the formulation is a dry powder formulation. A dry powder formulation of a macrocyclic cavity-containing compound can be formulated with additives, such as mannitol, raffinose, sucrose, maltose, lactose, glucose, xylitol, sorbitol, polyethylene glycol, biodegradable polymers (e.g. poly(lactic-co-glycolic acid)), magnesium stearate, lipids (e.g. DPPC, DSPC, DMPC), amino acids (e.g. isoleucine, trileucine, leucine, valine, phenylalanine), surfactants (e.g. poloxamer) and/or adsorption enhancers (e.g. bile salts, sodium citrate).

In one embodiment, the dry powder formulation comprises trehalose, sodium citrate, leucine and a macrocyclic, cavity-containing compound. In one embodiment, the dry powder formulation comprises mannitol, sodium citrate, leucine and a macrocyclic, cavity-containing compound. In one embodiment, the dry powder formulation comprises raffinose, sodium citrate, leucine and a macrocyclic, cavity-containing compound. In one embodiment, the dry powder formulation comprises maltose, sodium citrate, leucine and a macrocyclic, cavity-containing compound. In one embodiment, the dry powder formulation comprises dipalmitoylphosphatidylcholine (DPPC), leucine and a macrocyclic, cavity-containing compound. In one embodiment, the dry powder formulation comprises DPPC, trehalose, leucine and a macrocyclic, cavity-containing compound. In one embodiment, the dry powder formulation comprises DPPC, mannitol, leucine and a macrocyclic, cavity-containing compound. In one embodiment, the dry powder formulation comprises DPPC, raffinose, leucine and a macrocyclic, cavity-containing compound. In one embodiment, the dry powder formulation comprises DPPC, maltose, leucine and a macrocyclic, cavity-containing compound. In one embodiment, the dry powder formulation comprises lactose and a macrocyclic, cavity-containing compound. In one embodiment, the dry powder formulation comprises lactose, mannitol and a macrocyclic, cavity-containing compound.

In addition, the above specified dry powder formulations may contain also an antimicrobial agent. Thus, in one embodiment, the dry powder formulation comprises trehalose, sodium citrate, leucine, a macrocyclic, cavity-containing compound and an antimicrobial agent. In one embodiment, the dry powder formulation comprises mannitol, sodium citrate, leucine, a macrocyclic, cavity-containing compound and an antimicrobial agent. In one embodiment, the dry powder formulation comprises raffinose, sodium citrate, leucine, a macrocyclic, cavity-containing compound and an antimicrobial agent. In one embodiment, the dry powder formulation comprises maltose, sodium citrate, leucine, a macrocyclic, cavity-containing compound and an antimicrobial agent. In one embodiment, the dry powder formulation comprises DPPC, leucine, a macrocyclic, cavity-containing compound and an antimicrobial agent. In one embodiment, the dry powder formulation comprises DPPC, trehalose, leucine, a macrocyclic, cavity-containing compound and an antimicrobial agent. In one embodiment, the dry powder formulation comprises DPPC, mannitol, leucine, a macrocyclic, cavity-containing compound and an antimicrobial agent. In one embodiment, the dry powder formulation comprises DPPC, raffinose, leucine, a macrocyclic, cavity-containing compound and an antimicrobial agent. In one embodiment, the dry powder formulation comprises DPPC, maltose, leucine, a macrocyclic, cavity-containing compound and an antimicrobial agent. In one embodiment, the dry powder formulation comprises lactose, a macrocyclic, cavity-containing compound and an antimicrobial agent. In one embodiment, the dry powder formulation comprises lactose, mannitol, a macrocyclic, cavity-containing compound and an antimicrobial agent.

The present invention relates to an inhalable formulation comprising a macrocyclic, cavity-containing compound as a biologically active ingredient for use in abolishing or reducing virulence of a microbe in a subject having an infection caused by the microbe or being at risk of such an infection. The present invention relates also to an inhalable formulation comprising a macrocyclic, cavity-containing compound as a biologically active ingredient for use in inhibiting and/or treating and/or preventing a microbial infection in a subject having a microbial infection or being at risk of a microbial infection. In addition, the present invention relates to an inhalable formulation comprising a macrocyclic, cavity-containing compound and an antimicrobial agent as biologically active ingredients for use in abolishing or reducing virulence of a microbe in a subject having an infection caused by the microbe or being at risk of such an infection. The present invention relates to an inhalable formulation comprising a macrocyclic, cavity-containing compound and an antimicrobial agent as biologically active ingredients for use in inhibiting and/or treating and/or preventing a microbial infection in a subject having a microbial infection or being at risk of a microbial infection.

Further, the present invention relates to a method of abolishing or reducing virulence of a microbe in a subject by administering an inhalable formulation comprising a macrocyclic, cavity-containing compound as a biologically active ingredient to a subject having an infection caused by the microbe or being at risk of such an infection. The present invention also relates to a method of inhibiting and/or treating and/or preventing a microbial infection in a subject having a microbial infection or being at risk of a microbial infection by administering an inhalable formulation comprising a macrocyclic, cavity-containing compound as a biologically active ingredient to the subject. The present invention relates to a method of abolishing or reducing virulence of a microbe in a subject by administering an inhalable formulation comprising a macrocyclic, cavity-containing compound and an antimicrobial agent as biologically active ingredients to a subject having an infection caused by the microbe or being at risk of such an infection. The present invention relates also to a method of inhibiting and/or treating and/or preventing a microbial infection in a subject having a microbial infection or being at risk of a microbial infection by administering an inhalable formulation comprising a macrocyclic, cavity-containing compound and an antimicrobial agent as biologically active ingredients to the subject.

The inhalable formulations can be used for the treatment of local infections as well as systemic infections. In addition, the inhalable formulations can be used for the prevention of local infections as well as systemic infections. In one embodiment, the inhalable formulation is suitable for the treatment of a local infection. In one embodiment, the infection is a pulmonary infection. In one embodiment, the microbial infection is a systemic infection. In one embodiment, the microbial infection relates to a disease or a disorder that increases risk of a microbial infection in a subject. For instance, lower respiratory tract infections affect patients with cystic fibrosis, non-cystic fibrosis bronchiectasis, ventilator-associated pneumonia, hospital-acquired pneumonia. In one embodiment, the microbial infection relates to cystic fibrosis. In one embodiment, the inhalable formulation is suitable for the treatment of a systemic infection. In one embodiment, the inhalable formulation is suitable for the treatment of an acute infection. In one embodiment, the inhalable formulation is suitable for the treatment of an acute local infection. In one embodiment, the inhalable formulation is suitable for the treatment of an acute systemic infection. In one embodiment, the inhalable formulation is suitable for the treatment of a sub-acute infection. In one embodiment, the inhalable formulation is suitable for the treatment of a sub-acute local infection. In one embodiment, the inhalable formulation is suitable for the treatment of a sub-acute systemic infection. In one embodiment, the inhalable formulation is suitable for the treatment of a chronic infection. In one embodiment, the inhalable formulation is suitable for the treatment of a chronic local infection. In one embodiment, the inhalable formulation is suitable for the treatment of a chronic systemic infection. In one embodiment, the microbial infection is tuberculosis, sepsis infection, meningitis, bladder infection, wound infection or food poisoning.

In one embodiment, the microbial infection is caused by bacteria. In one embodiment, the microbial infection is caused by a bacterium that is resistant against the major antimicrobial agents typically used in the treatment of the infections caused by said bacterium. In one embodiment, the microbial infection is caused by a bacterium that has developed multiple drug resistance to broad-spectrum antibiotics. In one embodiment, the microbial infection is caused by Gram-positive bacteria. In one embodiment, the microbial infection is caused by bacteria belonging to genera Staphylococcus. In one embodiment, the microbial infection is caused by Staphylococcus aureus. In one embodiment, the microbial infection is caused by Gram-negative bacteria. In one embodiment, the microbial infection is caused by bacteria belonging to genera Pseudomonas, Acinetobacter, Vibrio, Enterobacter, Escherichia, Kluyvera, Salmonella, Shigella, Helicobacter, Haemophilus, Proteus, Serratia, Moraxella, Stenotrophomonas, Bdellovibrio, Campylobacter, Yersinia, Morganella, Neisseria, Rhizobium, Legionella, Klebsiella, Citrobacter, Cronobacter, Ralstonia, Xylella, Xanthomonas, Erwinia, Agrobacterium, Burkholderia, Pectobacterium, Pantoea, Acidovorax or an other genus of the family Enterobacteriaceae. In one embodiment, the microbial infection is caused by bacteria belonging to genera Pseudomonas. In one embodiment, the microbial infection is caused by bacteria belonging to genera Acinetobacter. In one embodiment, the microbial infection is caused by bacteria belonging to genera Vibrio. In one embodiment, the microbial infection is caused by bacteria belonging to genera Yersinia. In one embodiment, the microbial infection is caused by bacteria belonging to genera Rhizobium. In one embodiment, the microbial infection is caused by bacteria belonging to genera Klebisella. In one embodiment, the microbial infection is caused by bacteria belonging to genera Burkholderia. In one embodiment, the microbial infection is caused by Pseudomonas aeruginosa, Acinetobacter baumannii, Vibrio cholera, Vibrio fischeri, Yersinia pestis, Rhizobium leguminosarum or Klebisella pneumoniae. In one embodiment, the microbial infection is caused by Pseudomonas aeruginosa. In one embodiment, the microbial infection is caused by Acinetobacter baumannii. In one embodiment, the microbial infection is caused by Vibrio cholera. In one embodiment, the microbial infection is caused by Vibrio fischeri. In one embodiment, the microbial infection is caused by Yersinia pestis. In one embodiment, the microbial infection is caused by Rhizobium leguminosarum. In one embodiment, the microbial infection is caused by Klebisella pneumoniae. In one embodiment, the microbial infection is a systemic infection. In one embodiment, the microbial infection is a local and/or a pulmonary infection. In one embodiment, the microbial infection is a chronic infection. In one embodiment, the microbial infection is a sub-acute infection. In one embodiment, the infection is an acute infection or the infection is caused by planktonic microbes.

In one embodiment, the subject is a human or an animal.

The inhalable or pulmonary formulation of the present invention can be prepared by techniques known in the art. The inhalable or pulmonary formulation of the present invention can be administered by devices and/or techniques known in the art. Example for these devices are dry powder inhalers, metered dose inhalers and nebulizers. The formulation can be in liquid or in powder form, for example. In one embodiment, the formulation is a liquid formulation. In one embodiment, the formulation is a liquid formulation suitable for administration by liquid based delivery systems such as a nebulizer. In one embodiment, the formulation is a dry powder formulation. In one embodiment, the formulation is a dry powder formulation suitable for administration by or a dry powder inhaler (DPI). However, local drug efficacy can only be achieved by correct aerodynamic properties of the dry powder. In one embodiment, the formulation is in the form of microparticles for inhalation. In one embodiment, the particles have mass median aerodynamic diameter (MMAD) in the range of from 1 to 5 μm, in order to pass through the mouth, throat and conducting airways towards alveoli. In one embodiment, the MMDA of the microparticles is in the range of 1-2 μm. The formulation optionally contains necessary pharmaceutically acceptable additives and/or ingredients, such as fillers, diluents and/or adju-vants.

The dry powder formulations of the present invention have shown very good aerosolization properties. The formulations enable a macrocyclic cavity-containing compound, such as pillar[5]arene to be delivered to the targeted area. The formulations also enable correct dosing of the macrocyclic cavity-containing compound. A dry powder formulation of pillar[5]arene having trehalose, leucine and sodium citrate as additives is suitable for inhalation/pulmonary delivery providing high delivery rate to targeted lung tissues. It has no toxicity. Further, it is stable after the processing. The following examples are given to further illustrate the invention without, however, restricting the invention thereto.

### EXAMPLES

### Example 1—Dry Powder Formulations

For dry powder formulations of macrocyclic, cavity containing compounds pillar[5]arene, cucurbit[6]uril and α-cyclodextrin, 18-crown-6, 15-crown-5, γ-cyclodextrin were dissolved in deionized water together with the excipients, trehalose, raffinose, mannitol (as bulking and stabilizing excipient), sodium citrate (as absorption enhancer) and l-leucine. Final precursor solution was directly used in the aerosol process using the aerosol flow reactor method described in Eerikäinen et al., 2003; Lähde et al., 2008; and Raula et al., 2008a. Briefly, droplets were formed from ul-trasonic nebulizer and nitrogen gas laminar flow of 20 l/min carried the droplets in the tube (105 cm long and 3 cm wide). Temperature was set to 50° C. and the Reyn-olds number was 800. The residence time of the particles in this section was 2 seconds. Aerosols are then carried into the secondary section of the reactor where the aluminium alloy tube 7 cm long with 37 holes with the diameter of 3 mm that is heated up to 230° C. The residence time of the particles in this secondary section was 0.08 seconds. After the heated sections of the reactor, particles were carried into the porous tube where a large volume of gas (80 l/min) with turbulent flow cooled the carried particles rapidly. Particles were separated and collected in a cyclone. Prepared particles were stored over silica in a desiccator at room temperature (0-1% of relative humidity, 20° C.±4° C.) for further analysis. Particle size distributions in the gas phase were determined with an electrical low-pressure impactor, ELPI (Dek-ati LTD., Finland).

### Particle Morphology

The morphology of the particles was imaged with scanning electron microscope (SEM, Zeiss Sigma VP) at an acceleration voltage of 1.5-3 kV. The samples were coated with sputtered platinum or gold in order to stabilize them under the electron beam and to enhance image contrast.

### Aerosolization of the Particles

The aerosolization of the particles was studied using an inhalation simulator developed previously and described in Kauppinen et al., 2002; Kurkela et al., 2002, and Raula et al., 2009, wherein the perating principles have been established and the applicability for analysing the aerosolization of particles has been demonstrated. Briefly, the inhalation simulation was created by using pressurized air gas and vacuum. The inhalation profiles were simulated as a fast inhalation was achieved in 2 seconds. One inhalation profile was measured with previously mentioned settings for 8 seconds. Commercially available Easyhaler inhaler device was filled with approximately 1 g of the peptide particles. The doses were administered with 10 repetitions by pressure drops over the inhaler, which were adjusted to 2 kPa and 4 kPa. The pressure drops corresponded to the inspiration flow rates of 40 L/min and 55 L/min for Easyhaler. Powder emission from the inhaler was detected by weighing the inhaler before and after each inhalation. The particles were deposited on stages of a Berner-type low pressure impactor, BLPI. The stage aerodynamic cut-off diameters of BLPI is ranging from 0.03 to 15.61 μm and final mass distributions of each stage was measured gravimetrically as disclosed in Hillamo, R. E., Kauppinen, E. I., 1991. Mass median aerodynamic diameters (MMAD) and geometric standard deviations (GSD) of the deposited powders were determined according to the following formulas where mᵢ is the mass fraction of particles on the collection stage and M is the sum of mass fractions. Fine particle fractions (FPF, geometric mean diameter Dg≤5.5 μm) were expressed with reference to the emitted dose (ED):

$$MMAD = exp ( \frac{\sum ( {m}_{i} ln {D}_{i} )}{M} )$$

$$GSD = exp ( {( \frac{\sum ( {m}_{i} {{D}_{i}^{3} ( ln {D}_{i} - ln MMAD )}^{2}}{\sum ( {m}_{i} {D}_{i}^{3} ) - 1} )}^{1 / 2} )$$

One formulation for each of the macrocyclic, cavity-containing compound was prepared. In summary, all formulations have exhibited high aerosolization performance. SEM images have shown separate wrinkled microparticles for both formulations, and morphology did not differ distinctively from each other. Both formulations are suitable for pulmonary delivery of these molecules. Inhalation performance results are shown in Table 1.

TABLE 1

| | | | FPF | | |
|---|---|---|---|---|---|
| | ED | | (%, Dg | MMAD | |
| Samples | mg/dose | CV_{ED} | ≤5.5 μm | (μm) | GSD |
| Pillar[5]arene-Tre- | 2.30 | 0.17 | 50 | 1.40 | 1.79 |
| Leu-NaCit | | | | | |
| Cucurbit[6]uril-Tre- | 2.30 | 0.42 | 41 | 1.86 | 1.67 |
| Leu-NaCit | | | | | |
| α-Cyclodextrin-Tre- | 3.84 | 0.23 | 55 | 1.70 | 1.72 |
| Leu-NaCit | | | | | |
| 18-Crown-6-Colistin- | 2.32 | 0.18 | 51 | 1.40 | 1.52 |
| Tre-Leu-NaCit | | | | | |
| 15-Crown-5-Amika- | 2.35 | 0.24 | 50 | 1.54 | 1.43 |
| cin-Raf-Leu-NaCit | | | | | |
| γ-cyclodextrin-Cipro- | 2.20 | 0.23 | 42 | 1.52 | 1.56 |
| floxacin-Man-Leu- | | | | | |
| Na-Cit | | | | | |

Inhalation flow is 4 kPa=55 L/min. ED is average emitted dose; CVED is coef-ficient variation of emitted dose; Dg is geometric mean diameter; FPF is fine particle fraction; MMAD is mass median aerodynamic diameter; GSD is geometric standard deviation.

Pillar[5]arene containing dry powder formulations have shown 50% fine particle fraction (FPF), α-Cyclodextrin formulation had 55% FPF and Cucurbituril formulation had 41% FPF meaning that the percentage of the delivered dose could be delivered to the therapeutic area in the respiratory tract. In addition, dry powder formulations containing 18-Crown-6 and colistin, 15-Crown-5 and amikacin as well as γ-cyclodextrin and ciprofloxacin have shown 51% FPF, 50% FPF and 41% FPF, respectfully.

### Example 2

The interaction of P[5]a with lipopolysaccharides of P. aeruginosa, strain PA10 was measured using AUC. The results are shown in FIG. 11.

Analytical ultracentrifugation (AUC) is based on sedimentation of colloidal particles in a centrifugal field. During centrifugation particles move toward the bot-tom of the measuring cell. This movement leads to redistribution of particles along the measuring cell, which in turn can be expressed as a change in concentration along the measuring cell. The absorbance detector was used to follow the changes caused by centrifugation (absorbance is proportional to concentration). In simple words, in AUC the inventors measure how concentration changes during centrifugation, so they collect concentration profiles. Interaction between particles can be measured as a result of changes in the sedimentation profiles. In sedimentation velocity one uses high speed and collect the data (concentration profiles) during sedimentation process at different time points (many profiles).

The spectra of both P[5]a and LPS from P. aeruginosa were first analysed separately (see FIGS. 11a and b). At 305 nm, P[5]a displays a clear and steady sedimentation profile over time (as indicated by the Y-axis radius). At 305 nm, LPS does not display a sedimentation profile. Because only the sedimentation of P[5]a was detected at 305 nm, the inventors were certain that any changes observed in the sedimentation profile, come from interactions between P[5]a and other particles. The inventors then combined P[5]a together with LPS. LPS varies in compo-sition and has a molecular weight range between 10-20 kDa (see FIG. 11c). Rapid sedimentation of some particles were observed. Analysis of the molecular weight of the particles shows a large distribution of sizes (see FIG. 11d). The inventors observed a big first peak at low molecular weight (and a steady sedimentation spectrum in FIG. 11c, which is reminiscent of unbound P[5]a in FIG. 11a). This represents unbound P[5]a, meaning there was a large excess of unbound particles. The inventors also observed lower peaks across a large range of molecular weights. This indicates that P[5]a is capable of interacting with multiple LPS particles, to form polydisperse structures. https://www.sigmaaldrich.com/catalog/product/sigma/l9143?lang=&region=Fl

### Example 3

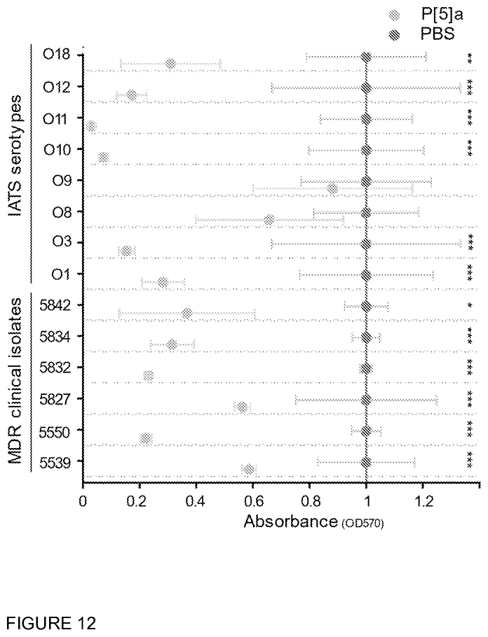

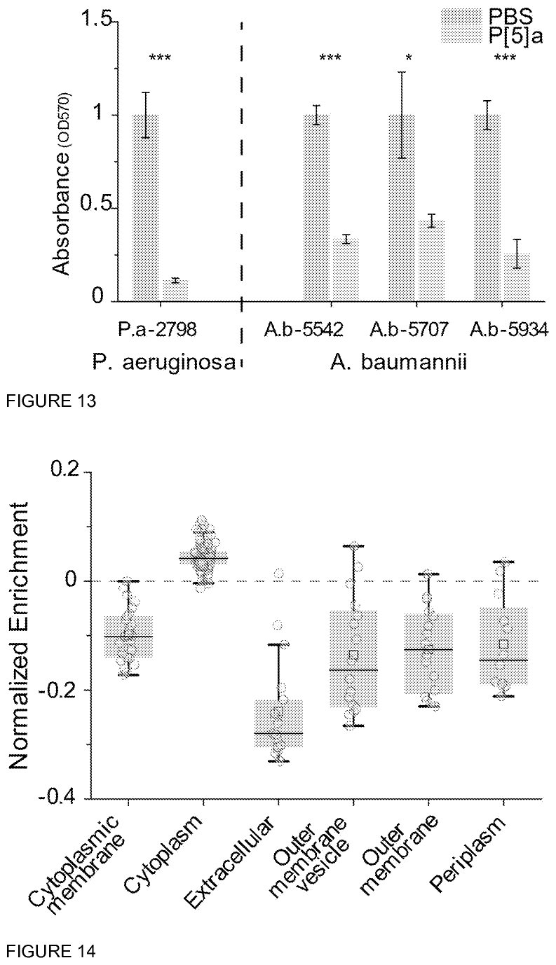

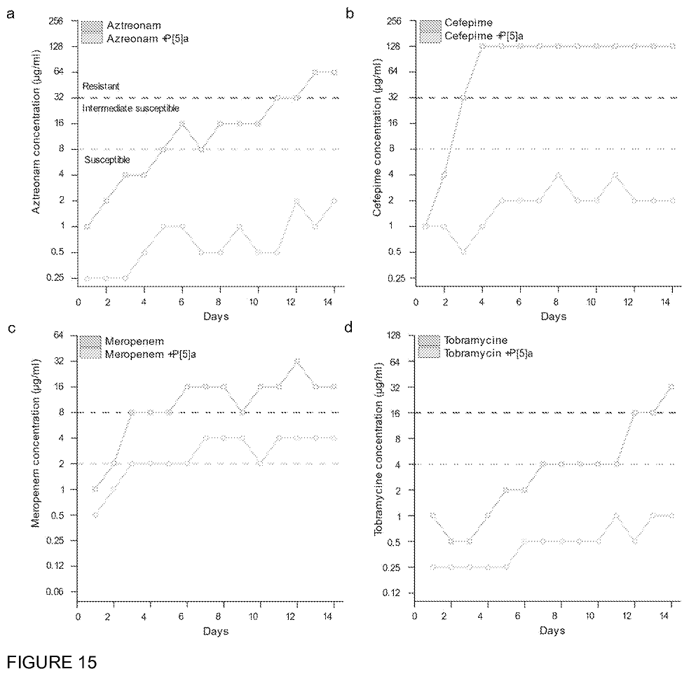

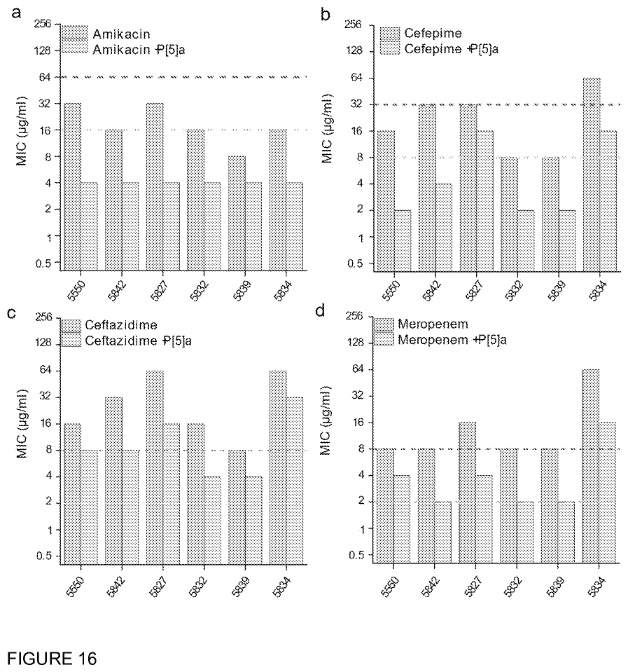

The effect of P[5]a on the formation of biofilm in the pathogenic Gram-negative bacteria Pseudomonas aeruginosa. Effects was tested against a variety of P. aeruginosa serotypes (IATS serotypes) as well as extensively multidrug resistant clinical isolates (resistance profiles and detailed strain information are provided in Table 2 below). The results are shown in FIG. 12. ***P<0.001; **P<0.01; *P 0.05.

TABLE 2

| | | Further | |
|---|---|---|---|
| | | specifi- | |
| code | Species | cation | Antibiotic resistance |
| 5542 | MDR- | | Resistant to Amikacin, Amoxicillin-clavulanate, Ampicillin, |
| | Acinetobacter | | Azithromycin, Cefalexin, Ertapenem, Levofloxacin, |
| | baumannii | | Meropenem, Minocycline, Piperacillin-tazobactam, |
| | | | Tigecycline, Tobramycin and Sulfamethoxazole |
| 5707 | MDR- | | MDR |
| | Acinetobacter | | |
| | baumannii | | |
| 5934 | MDR- | | MDR |
| | Acinetobacter | | |
| | baumannii | | |
| IATSO18 | Pseudomonas | ATCC | |
| | aeruginosa | 33365 | |
| IATSO12 | Pseudomonas | ATCC | |
| | aeruginosa | 33359 | |
| IATSO11 | Pseudomonas | ATCC | |
| | aeruginosa | 33358 | |
| IATSO10 | Pseudomonas | ATCC | |
| | aeruginosa | 33357 | |
| IATSO9 | Pseudomonas | ATCC | |
| | aeruginosa | 33356 | |
| IATSO8 | Pseudomonas | ATCC | |
| | aeruginosa | 33355 | |
| IATSO5 | Pseudomonas | ATCC | |
| (PA01) | aeruginosa | 33352 | |
| IATSO3 | Pseudomonas | ATCC | |
| | aeruginosa | 33350 | |
| IATSO1 | Pseudomonas | ATCC | |
| | aeruginosa | 33348 | |
| 5542 | Pseudomonas | | MDR Intermediate susceptible to amikacin 16 ug, resistant to |
| | aeruginosa | | cefepime 32 ug, ceftazidime 32 ug and meropenem 8 ug |
| 5834 | Pseudomonas | | MDR Intermediate susceptible to amikacin 16 ug, resistant to |
| | aeruginosa | | cefepime 64 ug, ceftazidime 64 ug and meropenem 64 ug |
| 5832 | Pseudomonas | | MDR intermediate susceptible to amikacin 16 ug and |
| | aeruginosa | | to cefepime 8 ug, resistant to ceftazidime 16 ug and |
| | | | meropenem 8 ug, resistant to ciprofloxacin |
| 5827 | Pseudomonas | | MDR intermediate susceptible to amikacin 32 ug, resistant to |
| | aeruginosa | | cefepime 32 ug, ceftazidime 64 ug and meropenem 16 ug |
| 5550 | Pseudomonas | | MDR Intermediate susceptible to amikacin 32 ug |
| | aeruginosa | | and to cefepime 16 ug, resistant to ceftazidime 16 ug |
| | | | and meropenem 8 ug |
| 5539 | Pseudomonas | | Susceptible to Amikacin 8 ug, intermediate to Cefepime 8 ug, |
| | aeruginosa | | resistant to ceftazidime 8 ug and meropenem 8 ug and |
| | | | imipenem, cefurixome, pipiracillin-tazobactam, |
| | | | ciprofloxacin and tobramycin |
| 2798 | Pseudomonas | ATCC | Susceptible to Amikacin & Colistin, Intermediate susceptible |
| | aeruginosa | 2798 | to Aztreonam & Cefepime, Resistant to Ceftazidime, |
| | | | Cirpofloxacin, Doripenem, Imipenem, Meropenem, |
| | | | Levofloxacin & Piperacillin-tazobactam |

### REFERENCES

Eerikäinen, H., Watanabe, W., Kauppinen, E. I., Ahonen, P. P., 2003. Aerosol flow reactor method for synthesis of drug nanoparticles. Eur. J. Pharm. Biopharm. 55, 357-360.
Lande, A., Raula, J., Kauppinen, E. I., 2008. Simultaneous synthesis and coating of salbutamol sulphate nanoparticles with L-leucine in the gas phase, Int. J. Pharm., 358, 256-262.
Lande, A., Raula, J., Kauppinen, E. I., 2008. Production of L-leucine nanoparticles under various conditions using an aerosol flow reactor method, J. Nanomat., Arti-cle ID 680897, Raula, J., Lande, A., and Kauppinen, E. I., 2008. A novel gas phase method for the combined synthesis and coating of pharmaceutical particles. Pharm. Res., 25, 242-245.
Raula, J., Kuivanen, A., Lande, A., Kauppinen, E. I., 2008. Gas-phase synthesis of L-leucine-coated micrometer-sized salbutamol sulphate and sodium chloride parti-Iles, Powder Technol., 187, 289-297.
Raula, J., Lande, A., Kauppinen, E. I., 2009. Aerosolization behavior of carrier-free L-leucine coated salbutamol sulphate powders, Int. J. Pharm., 365, 18-25. Kauppinen, E., Kurkela, J., Brown, D., Jokiniemi, J., Mattila, T., 2002. Method and apparatus for studying aerosol sources. WO 02/059574.
Kurkela, J. A., Kauppinen, E. I., Brown, D. P., Jokiniemi, J. K., Muttonen, E., 2002. A new method and apparatus for studying performance of inhalers. In: Dalby, R. N., Byron, P. R., Peart, J., Farr, S. J. (Eds.), Respiratory Drug Delivery VIII. DavisHor-wood Intl Publishing, Raleigh, N.C., pp. 791-794.
Hillamo, R., Kauppinen, E. I., 1991. On the performance of the Berner low pressure impactor. Aerosol Sci. Technol. 14, 33-47.

1. An inhalable formulation comprising a macrocyclic, cavity-containing compound as a biologically active ingredient and a pharmaceutically acceptable additive, and optionally an antimicrobial agent.

2. (canceled)

3. (canceled)

4. (canceled)

5. A method of inhibiting/treating/preventing a microbial infection in a subject having a microbial infection or being at risk of a microbial infection by administering an inhalable formulation comprising a macrocyclic, cavity-containing compound as a biologically active ingredient and optionally an antimicrobial agent to the subject.

6. (canceled)

7. The inhalable formulation of claim 1, wherein the macrocyclic cavity-containing compound is selected from pillararenes, cyclodextrins, cucurbiturils, crown ethers, calixarenes and/or salts thereof.

8. The inhalable formulation according to claim 7, wherein the compound is a pillar arene or a salt thereof, optionally a pillar[5]arene or a salt thereof.

9. (canceled)

10. The inhalable formulation according to claim 7, wherein the compound is selected from alpha-cyclodextrins, gamma-cyclodextrins and/or salts thereof.

11. The inhalable formulation according to claim 7, wherein the compound is a cucurbituril, optionally cucurbit[6]uril.

12. (canceled)

13. The method according to claim 5, wherein the microbial infection is an acute infection, a sub-acute infection or a chronic infection.

14. The method according to claim 5, wherein the microbial infection is a systemic infection or a local infection.

15. The method according to claim 5, wherein the microbial infection is caused by a Gram-positive bacteria, optionally belonging to genera Staphylococcus.

16. (canceled)

17. The method according to claim 5, wherein the microbial infection is caused by a Gram-negative bacteria, optionally belonging to genera Pseudomonas, Acinetobacter, Vibrio, Enterobacter, Escherichia, Kluyvera, Salmonella, Shigella, Helicobacter, Haemoph ilus, Proteus, Serratia, Moraxella, Stenotrophomonas, Bdellovibrio, Campylobacter, Yersinia, Morganella, Neisseria, Rhizobium, Legionella, Klebsiella, Citrobacter, Cronobacter, Ralstonia, Xylella, Xanthomonas, Agrobacterium, Burkholderia, Pectobacterium, Pantoea, Acidovorax or any other genus of the family Enterobacteriaceae.

18. (canceled)

19. The inhalable formulation according to claim 1, wherein the antimicrobial agent is selected from b-lactams, such as imipenem and meropenem, aminoglycosides, such as amikacin and tobramycin, fluoroquinolones, such as levofloxacin, quinolones, macrolides, novobiocin, tetracyclines, chloramphenicol, ethidium bromide, cephalosporins such as cefepime, ceftazidime and ceftriaxone, and colistin.

20. The inhalable formulation according to claim 1, wherein the formulation is a dry powder formulation.

21. The inhalable formulation according to claim 1, wherein the pharmaceutically acceptable additive is selected from leucine, mannitol, maltose, raffinose, lactose, trehalose, sodium citrate and/or DPPC.

22. The method of claim 5, wherein the macrocyclic cavity-containing compound is selected from pillararenes, cyclodextrins, cucurbiturils, crown ethers, calixarenes and/or salts thereof.

23. The method according to claim 22, wherein the compound is a pillar arene or a salt thereof, optionally a pillar[5]arene or a salt thereof.

24. The method according to claim 22, wherein the compound is selected from alpha-cyclodextrins, gamma-cyclodextrins and/or salts thereof.

25. The method according to claim 22, wherein the compound is a cucurbituril, optionally cucurbit[6]uril.

26. The method according to claim 5, wherein the antimicrobial agent is selected from β-lactams, such as imipenem and meropenem, aminoglycosides, such as amikacin and tobramycin, fluoroquinolones, such as levofloxacin, quinolones, macrolides, novobiocin, tetracyclines, chloramphenicol, ethidium bromide, cephalosporins such as cefepime, ceftazidime and ceftriaxone, and colistin.
